(19) United States
(12) Patent Application Publication
(10) Pub. No.: US 2021/0308299 A1
(43) Pub. Date: Oct. 7, 2021

(54) DISINFECTING AND/OR STERILIZING DEVICE AND METHODS OF MAKING AND USING THE SAME

(71) Applicant: Torkild Benn Vennesland, Carolina Beach, NC (US)

(72) Inventor: Torkild Benn Vennesland, Carolina Beach, NC (US)

(21) Appl. No.: 17/184,711

(22) Filed: Feb. 25, 2021

Related U.S. Application Data
(60) Provisional application No. 63/006,560, filed on Apr. 7, 2020.

(51) Int. Cl. A61L 2/10

(52) U.S. Cl. CPC A61L 2/10; A61L 2202/20; A61L 2202/122; A61L 2202/11

(57) ABSTRACT

The presently disclosed subject matter is directed to a device configured to sterilize and/or disinfect one or more flat objects, such as (but not limited to) credit cards, identification cards, papers, business cards, currency, passports, and the like. The device includes a housing that comprises an internal compartment sized and shaped to house one or more objects. The internal compartment includes a plurality of bulbs (e.g., ultraviolet bulbs) positioned such that the entire surface of an object housed within the internal compartment is exposed to the bulbs. One or more objects can be at least partially positioned within the interior of the compartment via inlet and outlet slots. Thus, in use, an object enters the device interior via an inlet slot where it is exposed to the emitting bulbs. The sterilized and/or disinfected object then exits the device interior through exit slot where it can be used.

### CROSS-REFERENCE TO RELATED APPLICATIONS

This application claims priority to U.S. Provisional Patent Application No. 63/006,560 filed Apr. 7, 2020, the entire content of which is hereby incorporated by reference.

### TECHNICAL FIELD

The presently disclosed subject matter is directed to a disinfecting and/or sterilizing device that can be used with a variety of flat objects (e.g., currency, credit cards, documents). The presently disclosed subject matter further includes methods of making and using the device.

### BACKGROUND

The spread of viruses and other harmful microorganisms presents a challenge to protecting individuals in a society where people live and work in close proximity to each other. For example, credit cards, currency, identification cards, and the like are frequently used on a daily basis, such as in stores, restaurants, at work, and the like. The items are commonly handled by the owner, as well as mutual credit card machines, retail employees, and the like. As a result, viruses and other harmful microorganisms can be easily spread from person to person. Current methods of sterilizing and disinfecting objects include the use of liquid chemicals and similar disinfecting sprays. However, these methods are unsuitable for portable, flat objects such as currency, credit cards, documents, and the like. Particularly, hard plastic items (such as credit cards) must be wetted and dried between uses, which can be cumbersome and time consuming. Fibrous materials such as currency are typically disinfected by washing, which is laborious and inefficient. As a result, the risk of cross-contamination during use of everyday objects remains prevalent. It would therefore be beneficial to provide a device capable of quickly and easily disinfecting and/or sterilizing flat and portable objects.

### SUMMARY

In some embodiments, the presently disclosed subject matter is directed to a device for sterilizing and/or disinfecting an object. Particularly, the device comprises a housing and a plurality of ultraviolet bulbs positioned within the interior compartment of the housing, adjacent to the pathway. The housing is defined by a front face and an opposing rear face; a top face and an opposing bottom face; and a first side face and a second side face, wherein each side face includes a slot that provides access to the interior compartment. The housing further includes a pathway that extends from the first side face slot to the second side face slot within the interior compartment.

In some embodiments, the device comprises a plurality of ultraviolet bulbs positioned above and below the pathway.

In some embodiments, the external surface of each slot includes a shielding mechanism.

In some embodiments, the interior compartment includes a coating that at least partially blocks or reduces ultraviolet radiation at or adjacent to the slots.

In some embodiments, each ultraviolet bulb emits radiation with a wavelength of about 100-400 nanometers.

In some embodiments, each ultraviolet bulb emits UV-A light, UV-B light, UV-C light, or combinations thereof.

In some embodiments, each ultraviolet bulb has a wavelength sufficient to kill about 80-100 percent of one or more viruses, bacteria, molds, or fungi.

In some embodiments, at least one ultraviolet bulb is positioned above the pathway, below the pathway, or both.

In some embodiments, the plurality of ultraviolet bulbs are positioned in an arcuate pattern to at least partially surround the pathway on all sides.

In some embodiments, the plurality of bulbs include varying levels of ultraviolet light.

In some embodiments, the device further includes a plurality of rollers positioned directly above, below, or both above and below the pathway.

In some embodiments, the rollers are electromechanical rollers (e.g., including both electronic and mechanical components that can move during operation, such as an electric drive and/or actuator coupled with mechanical components that transmit electrical energy into mechanical motion).

In some embodiments, the presently disclosed subject matter is directed to a method of decontaminating and/or sterilizing an object. Particularly, the method comprises positioning the object within a slot of a decontaminating or sterilizing device. The device comprises a housing defined by: a front face and an opposing rear face; a top face and an opposing bottom face; a first side face and a second side face, wherein each side face includes a slot that provides access to the interior compartment; and a pathway that extends from the first side face slot to the second side face slot within the interior compartment. The device further includes a plurality of ultraviolet bulbs positioned within the interior compartment of the housing. The method comprises passing the object along the pathway of the device interior compartment, wherein the object is exposed to the ultraviolet bulbs on all sides. The method includes removing the object from the opposing slot, wherein the object is decontaminated or sterilized on all sides.

In some embodiments, the object is passed along the pathway using one or more rollers positioned along the pathway.

In some embodiments, the object can pass from the slot on the first side face to the slot on the second side face and vice versa.

In some embodiments, the output profile of each ultraviolet bulb is varied as desired by the user. The “output profile” refers to the amount and/or intensity of the bulb output. The “output profile” can optionally also include the amount of time that the bulb is “on” (e.g., emitting UV light).

### BRIEF DESCRIPTION OF THE DRAWINGS

The previous summary and the following detailed descriptions are to be read in view of the drawings, which illustrate some (but not all) embodiments of the presently disclosed subject matter.

FIG. 1 is a perspective view of a disinfecting and/or sterilizing device in accordance with some embodiments of the presently disclosed subject matter.

FIG. 2a is a perspective view of a disinfecting and/or sterilizing device housing in accordance with some embodiments of the presently disclosed subject matter.

FIG. 2b is a cross-sectional view of a disinfecting and/or sterilizing device housing in accordance with some embodiments of the presently disclosed subject matter.

FIG. 2c is a front plan view of a device input or exit slot in accordance with some embodiments of the presently disclosed subject matter.

FIG. 2d is a front plan view of a device input or exit slot in accordance with some embodiments of the presently disclosed subject matter.

FIG. 3a is a cross-sectional view of a device housing in accordance with some embodiments of the presently disclosed subject matter.

FIG. 3b is a perspective view of a device housing in accordance with some embodiments of the presently disclosed subject matter.

FIG. 4 is a front plan view of a UV bulb in accordance with some embodiments of the presently disclosed subject matter.

FIG. 5a is a cross-sectional view of a disinfecting and/or sterilizing device comprising a parallel arrangement of bulbs in accordance with some embodiments of the presently disclosed subject matter.

FIG. 5b is a cross-sectional view of a disinfecting and/or sterilizing device comprising an offset arrangement of bulbs in accordance with some embodiments of the presently disclosed subject matter.

FIG. 5c is a cross-sectional view of a disinfecting and/or sterilizing device comprising an arcuate arrangement of bulbs in accordance with some embodiments of the presently disclosed subject matter.

FIG. 6a is a cross-sectional view of a disinfecting and/or sterilizing device comprising a plurality of rollers in accordance with some embodiments of the presently disclosed subject matter.

FIG. 6b is a cross-sectional view of a disinfecting and/or sterilizing device comprising a plurality of rollers that partially extend across the interior compartment pathway in accordance with some embodiments of the presently disclosed subject matter.

FIG. 6c is a cross-sectional view of a disinfecting and/or sterilizing device comprising a plurality of rollers that fully extend across the interior compartment pathway in accordance with some embodiments of the presently disclosed subject matter.

FIG. 6d is a cross-sectional view of a disinfecting and/or sterilizing device comprising a plurality of rollers positioned below the interior compartment pathway in accordance with some embodiments of the presently disclosed subject matter.

FIG. 6e is a cross-sectional view of a disinfecting and/or sterilizing device comprising a plurality of rollers positioned above the interior compartment pathway in accordance with some embodiments of the presently disclosed subject matter.

FIG. 7a is a side plan view of a target object configured as a credit card in accordance with some embodiments of the presently disclosed subject matter.

FIG. 7b is a side plan view of a target object configured as a document in accordance with some embodiments of the presently disclosed subject matter.

FIG. 8a is a perspective view of one method of inserting an object into a disinfecting and/or sterilizing device in accordance with some embodiments of the presently disclosed subject matter.

FIGS. 8b and 8c are cross-sectional views of passing a target object through a disinfecting and/or sterilizing device in accordance with some embodiments of the presently disclosed subject matter.

FIG. 8d is a cross-sectional view of one method of removing an object from a sterilizing and/or disinfecting device in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−0.1-20% from the specified amount, as such variations are appropriate in the disclosed packages and methods.

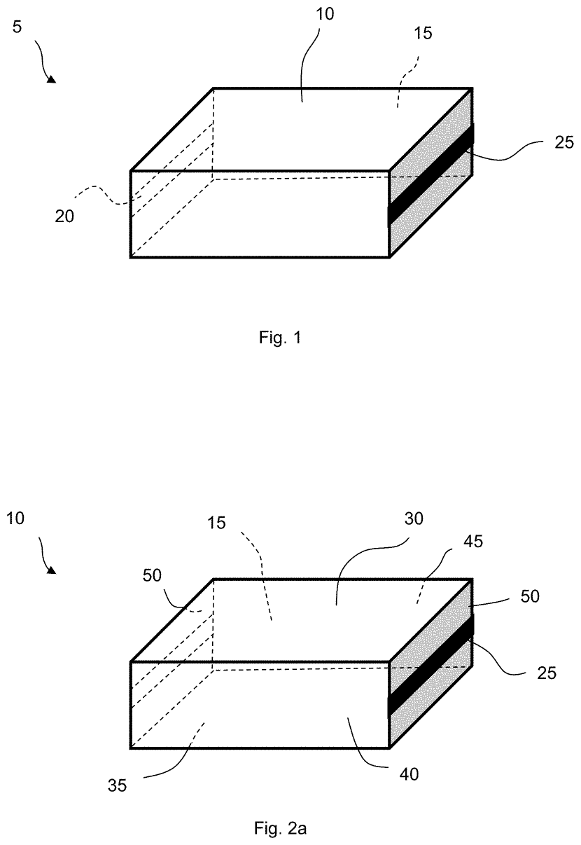

The presently disclosed subject matter is directed to a device configured to sterilize and/or disinfect one or more flat objects, such as (but not limited to) credit cards, identification cards, papers, business cards, paper money, passports, and the like. FIG. 1 illustrates one embodiment of device 5 comprising housing 10 comprising internal compartment 15 sized and shaped to house one or more objects. The internal compartment includes a plurality of bulbs (e.g., ultraviolet bulbs) positioned such that the entire surface of an object housed within the internal compartment is exposed to the bulbs. One or more objects can be at least partially positioned within the interior of compartment 15 via inlet and outlet slots 20, 25. Thus, in use, an object enters the device interior via inlet slot 20 where it is exposed to the emitting bulbs. The sterilized and/or disinfected object then exits the device interior through exit slot 25, as described in more detail herein below.

The term “disinfect” or “disinfecting” as used herein refers to the removal, inhibition, neutralization, and/or killing of microbial organisms on a surface. The term “sterilize” or “sterilizing” as used herein refers to the killing of all microbial organisms on a surface. It should be appreciated that the while the terms “sterilize” and “disinfect” have different scientific meanings, they are used interchangeably herein, as actual results of the presently disclosed subject matter can differ according to the nature of the targeted microorganism, the product being processed, and/or the methods used.

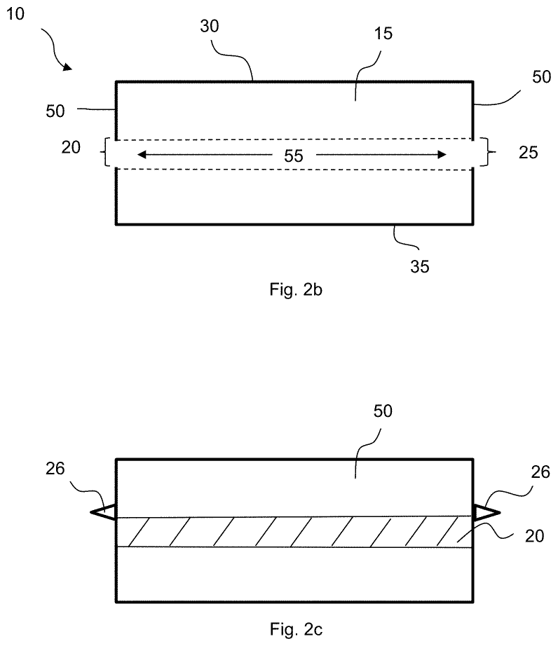

As set forth above, device 5 includes housing 10 that protects and contains the interior emitting bulbs. As illustrated in FIG. 2a, the housing is defined by top face 30, opposed bottom face 35, front and rear faces 40, 45, and a pair of side faces 50. The faces of the housing are interconnected to provide a secure exterior capable of supporting the contents of the interior compartment. Side faces 50 each comprise a slot that can be used to insert and/or remove an object to be disinfected and/or sterilized. The term “slot” broadly refers to any aperture positioned on a face of the housing that allows access to the housing interior compartment.

FIG. 2b illustrates one embodiment of housing 10 comprising input slot 20 and exit slot 25 with pathway 55 therebetween within the interior of the device. The input and exit slots can be sized and shaped to allow a target object to pass therethrough. For example, slots 20, 25 can have any desired shape, such as (but not limited to), square, rectangular, oval, round, triangular, pentagonal, hexagonal, abstract, and the like. It should be appreciated that the input and exit slots are shaped to ease insertion of an object into the device interior compartment, and to ensure that the object passes smoothly between or through the bulbs until the object exits the device. When the object is relatively small (e.g., a credit card), the input and exit slots can be small, such as about 1-3 inches in length and/or width. In other embodiments, the input and exit slots can be larger, such as about 6-12 inches (e.g., for documents) in length and/or width. The input and exit slots can be configured in about the same size and/or shape. Alternatively, the slots can differ with respect to size and/or shape.

In some embodiments, slots 20, 25 can extend the full length of side face 50, as shown in FIG. 2c. However, the presently disclosed subject matter is not limited and length 60 of the slot can extend about 40-99 percent of the length 61 of the side face (e.g., at least/no more than about 40, 45, 50, 55, 60, 65, 70, 75, 80, 85, 90, 95, 99 percent), as shown in FIG. 2d.

Slots 20, 25 can be configured on side face 50 of the housing using any conventional method, such as (but not limited to) the use of laser cutting, thermoforming, etching, sharp instruments (e.g., blades and the like), and other similar methods.

The device housing is designed to maintain the functional elements of the device safely and effectively. For example, certain wavelengths of ultraviolet radiation are harmful to humans and animals, and exposure can cause sunburn and eventually skin cancer. Exposure to the naked eye is also known to lead to temporary or permanent vision impairment by damaging the retina of the eye. For this reason, input and exit slots 20, 25 can be designed to limit the amount of ultraviolet radiation that exits the device to the external environment. Thus, in some embodiments, the slots can include a flap, overhang, or other shielding mechanism 26 to protect the user from ultraviolet radiation, as shown in FIG. 2c. Similarly, interior compartment of the housing can be coated with materials to reduce ultraviolet radiation that inadvertently escapes through the input and exit slots.

Housing 10 can be constructed from any rigid or semi-rigid material. The term “rigid” refers to a material that is stiff, unyielding, and typically unable to substantially give way under pressure without breaking. The term “semi-rigid” refers to a material that is rigid to some degree but is not inflexible. Suitable rigid or semi-rigid materials can include (but are not limited to) metal (e.g., stainless steel, copper), plastic, ceramics, concrete, stone, wood, or combinations thereof. In some embodiments, at least one material used to construct housing 10 can be impermeable to ultraviolet radiation, such as (but not limited to) stainless steel, chrome, and/or other metals. In other embodiments, the external surface of the housing can include a coating that is at least partially impermeable to ultraviolet radiation. In some embodiments, the coatings can be applied to at least a portion of the internal compartment (e.g., adjacent to the slots to reduce the amount of ultraviolet radiation that escapes via the slots). Suitable ultraviolet radiation-impermeable coatings can include (but are not limited to) resin, urethane, epoxy, and the like. The term “impermeable” refers to a material through which UV light cannot pass through (e.g., at least about 50, 55, 60, 65, 70, 75, 80, 85, 90, 95, 99, 99.9, or 100 percent of the UV light cannot pass through).

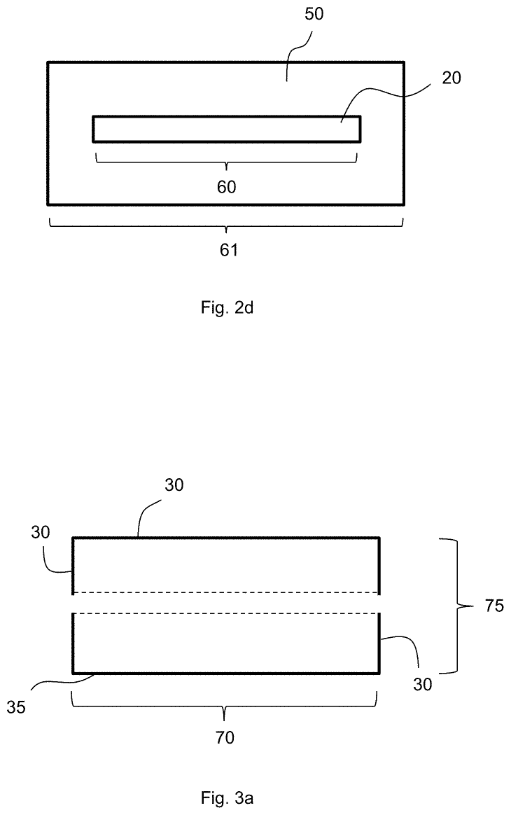

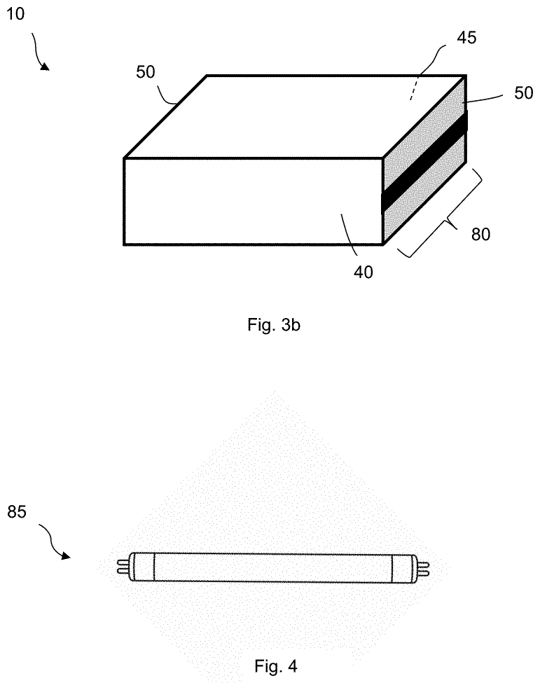

Housing 10 can have any desired length, width, and/or thickness. As shown in FIG. 3a, the housing can include length 70 of about 2-10 inches (e.g., at least/no more than about 2, 2.5, 3, 3.5, 4, 4.5, 5, 5.5, 6, 6.5, 7, 7.5, 8, 8.5, 9, 9.5, or 10 inches). The term “length” refers to the longest horizontal distance of the device (e.g., the longest horizontal distance spanning the distance between first and second side edges 50). Housing 10 can further include height of about 1-5 inches (e.g., at least/no more than about 1, 1.5, 2, 2.5, 3, 3.5, 4, 4.5, or 5 inches). The term “height” refers to the longest vertical distance of the device (e.g., the longest vertical distance spanning top face 30 to bottom face 35). The disclosed device also includes thickness 80 of about 1-10 inches (e.g., at least/no more than about 1, 1.5, 2, 2.5, 3, 3.5, 4, 4.5, 5, 5.5, 6, 6.5, 7, 7.5, 8, 8.5, 9, 9.5, or 10 inches). The term “thickness” refers to the longest distance that spans front face 40 to rear face 45, as shown in FIG. 3b. However, the length, height, and thickness of device 5 is not limited and can be configured outside the ranges given herein.

Housing 10 can have any desired cross-sectional shape, such as rectangular, square, rounded, abstract and the like.

The disclosed housing can be constructed using any conventional method. For example, the housing can be formed via thermoforming, welding, and the like, as would be well known in the art.

As set forth above, the housing includes interior compartment 15 that comprise one or more bulbs capable of emitting a disinfecting and/or sterilizing material. FIG. 4 illustrates one embodiment of bulb 85 that can be housed within the device interior. The term “bulb” refers to any element capable of emitting a disinfecting or sterilizing light, such as (but not limited to) ultraviolet radiation. Suitable ultraviolet bulbs can emit electromagnetic radiation with a wavelength of about 100-400 nm (e.g., at least/no more than about 100, 150, 200, 250, 300, 350, or 400 nm). While not bound by any theory, ultraviolet irradiation is believed to kill or inactivate microorganisms by destroying nucleic acids and disrupting their DNA, leaving them unable to perform vital cellular functions.

Bulbs 85 can comprise any UV light generating materials, such as (but not limited to) mercury bulbs, titanium dioxide, silver, copper, LED (light emitting diode) and the like. The term “bulb” therefore broadly refers to any device capable of generating UV light (e.g., LED). The UV bulbs are capable of producing at least one of UV-A, UV-B, and UV-C light wavelengths. The term “UV-A” refers to ultraviolet electromagnetic radiation with a wavelength from about 320 nm to about 400 nm and includes UV-A1 (from about 340 nm to about 400 nm) and UV-A2 (from about 320 nm to about 340 nm). The term “UV-B” refers to ultraviolet electromagnetic radiation with a wavelength from about 290 nm to about 320 nm. The term “UV-C” refers to ultraviolet electromagnetic radiation with a wavelength from about 200 nm to about 290 nm. Non-limiting examples of commercially available UV bulbs include non-xenon germicidal UV bulbs.

In some embodiments, the UV light is sufficient to kill and/or neutralize one or more harmful microorganisms. The harmful microorganisms can include (but are not limited to) bacteria (e.g., E. coli, septicaemia, tetanus, pneumonia, impetigo, tonsillitis, strep, legionella, staphylococcus, streptococcus, clostridium, and the like), viruses (e.g., coronavirus, influenza, Ebola, Marburg, rabies, HIV, and the like), molds (aspergillus, Trichoderma, Bipolaris, Chaetomium, Geotrichum, Alternaria, Penicillum, Memnoniella, Stachybotrys, and the like), fungi (Aspergillosis, Blastomycosis, Candidiasis, and the like), and combinations thereof. In some embodiments, about 50-100% of the microorganisms are killed or neutralized (e.g., at least/no more than about 50, 55, 60, 65, 70, 75, 80, 85, 90, 95, 99, 99.9, or 100 percent).

Bulb 85 can be configured in any desired shape and/or size and is not limited to the embodiment illustrated in the Figures. For example, the bulbs can be cylindrically shaped, although any shape or array that provides sufficient exposure to an object as it passes through the compartment pathway can be included.

The disclosed bulbs can be powered using any conventional methods, such as the use of a power cord, power supply, and/or batteries.

Device 5 can include any number of bulbs 85, such as (but not limited to) about 2-20. Thus, the interior compartment of the disclosed device can include about 2, 3, 4, 5, 6, 7, 8, 9, 10, 11, 12, 13, 14, 15, 16, 17, 18, 19, or 20 bulbs. However, the presently disclosed subject matter is not limited and device 5 can include more or fewer bulbs than provided in the given range.

In some embodiments, interior surfaces of housing 10 can be coated with one or more materials that increase ultraviolet radiation reflection to enhance the effectiveness of the sterilization process. Suitable coatings can include, but are not limited to, aluminium, foil, mirrored surfaces, and the like. Thus, the coatings can increase the exposure of bulbs 50 by about 25, 50, 100, 150, 200, 250, 300, 350, or 400 percent or more.

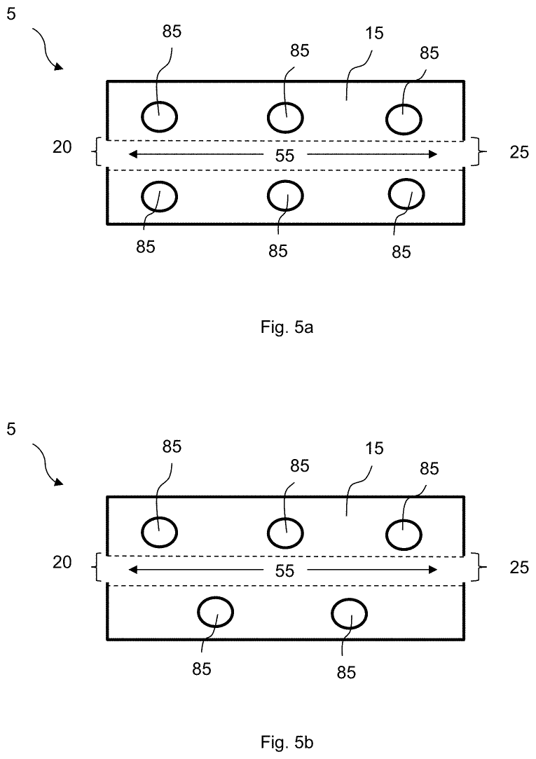

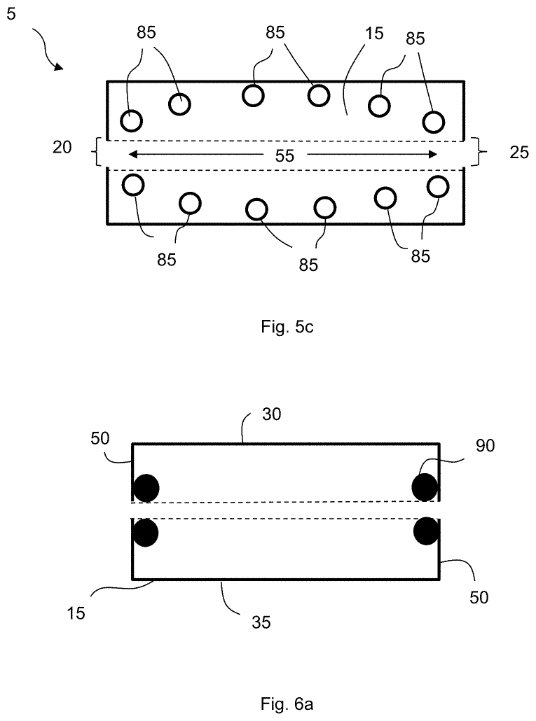

As shown in FIG. 5a, interior 15 of the housing includes pathway 55 between inlet and outlet slots 20, 25. An object to be disinfected passes from the inlet to the outlet along pathway 55. One or more bulbs 85 are be positioned above and/or below pathway 55 to ensure that all surfaces of the object are contacted by the disinfecting and/or sterilizing material (e.g., UV light). In this way, a direct light source is positioned to contact both surfaces and edges of the target flat object. The position of the upper bulbs (e.g., those positioned above pathway 55) can mirror the position of the lower bulbs, as shown in FIG. 5a. However, bulbs 85 can be offset as shown in FIG. 5b. It should be appreciated that device interior 35 can include any arrangement or configuration of bulbs 50.

For example, in some embodiments, it may be advantageous to include bulbs 85 in an arcuate pattern to surround at least a portion of the object to be sterilized when traveling along pathway 55, as shown in FIG. 5c. The term “arcuate” refers to a circular and or arched shape.

Each bulb 85 housed within the interior compartment of device 5 can have about the same output profile. Alternatively, the output profile of bulbs 85 can be varied as desired by the user (e.g., consumer, designer, manufacturer). For example, the bulbs closest to slots 25, 20 can have the least amount of ultraviolet radiation emission to ensure that the smallest amount of radiation possible extends through the slots. Similarly, the bulbs positioned farthest from the slots (e.g., those towards the middle of the interior compartment) can have the highest amount of UV emission. It should therefore be appreciated that the bulbs can include any desired configuration, such as random arrangement, side-by-side arrangement, sequential arrangement, array arrangement, clustered arrangement, parallel arrangement, and the like.

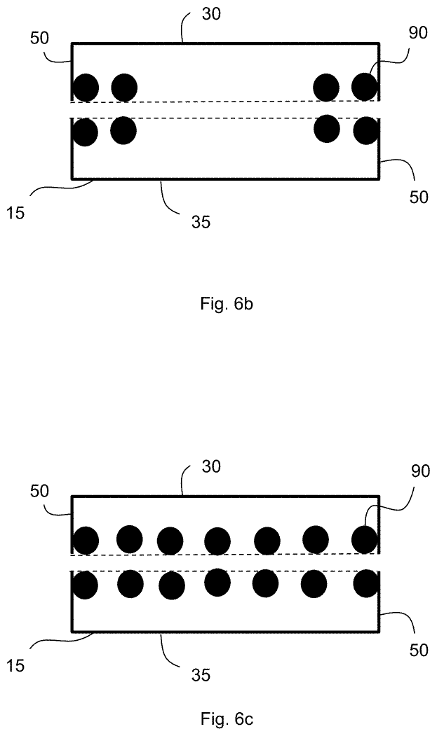

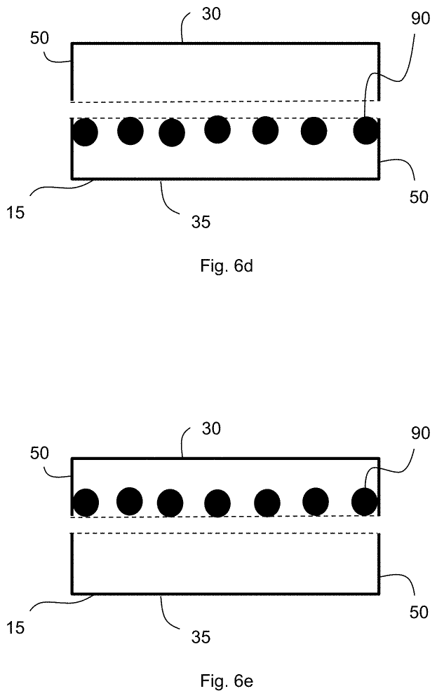

In some embodiments, the interior compartment 15 can include one or more rollers 90 to secure and move the object along pathway 55. Rollers 90 can include any mechanism to facilitate movement of the object within the interior compartment of device 5. The rollers can therefore include wheels, guides, and the like that engage the object to guide it horizontally into and out of slots 20, 25. In some embodiments, rollers 85 can be electromechanical rollers. The rollers can be positioned adjacent to the input and exit slots, as shown in FIG. 6a. In other embodiments, the rollers can fully or partially extend along the length of the pathway, as shown in FIGS. 6b and 6c.

Rollers 90 can be positioned above and below pathway 55, as illustrated in FIGS. 6a-6c. However, the presently disclosed subject matter is not limited and the rollers can be positioned only above or below the pathway, as shown in FIGS. 6d and 6e.

The device can include any number of rollers, such as about 1-50 (e.g., at least/no more than about 1, 5, 10, 15, 20, 25, 30, 35, 40, 45, or 50). However, the presently disclosed subject matter is not limited and the device can include greater than 50 rollers. It should be appreciated that rollers 90 can be configured in any size.

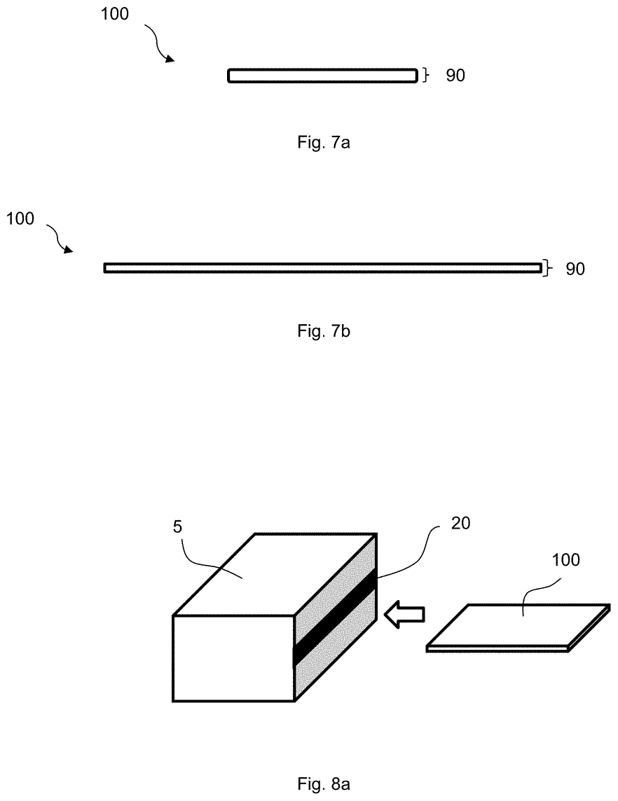

In use, an object to be decontaminated and/or sterilized by passing through the interior of device 5. The term “object” as used herein can refer to any object to be sterilized and/or disinfected using device 5. In some embodiments, object 100 is relatively flat, such as the credit card of FIG. 7a and the document of FIG. 7b, with thickness 95 of about 1, 0.75, 0.5, 0.25, 0.1, or 0.01 inches or less. Suitable objects can therefore include (but are not limited to) credit cards, gift cards, business cards, driver's licenses, passports, identification cards, social security cards, papers, brochures, pamphlets, currency (i.e., paper money), and the like.

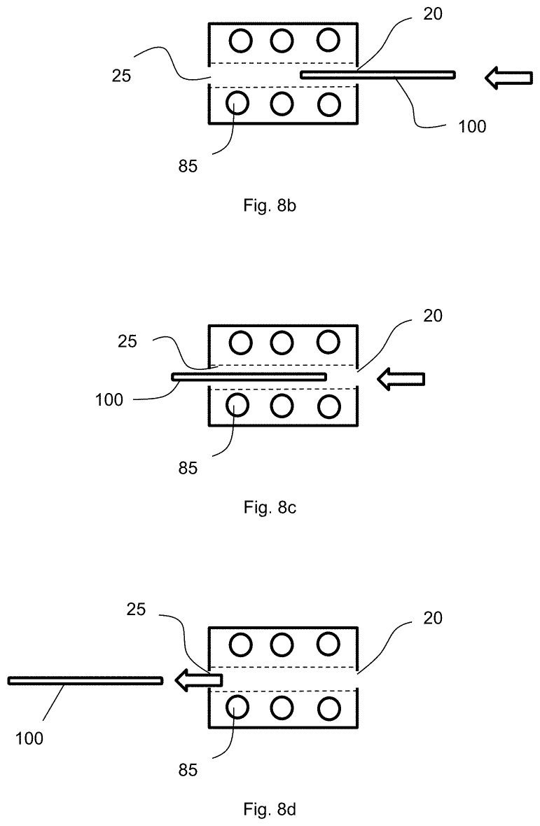

Object 100 is introduced into the interior compartment of the device through input slot 20 positioned on one side of the device as illustrated in FIG. 8a. The object is then passed completely through the interior compartment, from the input to the exit slot, along pathway 55, as shown in 8b. The object can be manually pushed along the pathway until it reaches exit 25, where the user can remove the object from the device. Alternatively, rollers 90 can be used to facilitate movement along pathway 55. While positioned within the interior compartment of device 5, object 100 is in contact with ultraviolet radiation from bulbs 85, as shown in FIG. 8c. In this way, the object is decontaminated and/or sterilized. In some embodiments, all surfaces of the object are decontaminated and/or sterilized via bulbs 85. The decontaminated and/or sterilized object can then exit the device through exit slot 25, as shown in FIG. 8d.

The object can pass through either direction of device 5, such as from inlet to exit slots or exit to inlet slots so long as the object proceeds completely through the interior compartment pathway in a single flow direction. In this way, movement of target object 100 is facilitated between two people handing the object (or different objects). For example, a credit can be passed through device 5 from the owner in a contaminated state. As the credit card exits the device, it has been decontaminated and/or sterilized such that the vendor can then run the clean credit card. After use, the vendor can again run the credit card through the device, decontaminating the object from any microorganisms that may be present after contact with the vendor and/or surrounding environment. The owner then reclaims the newly decontaminated and/or sterilized card as it exits the device slot and stores the card for later use.

Device 5 includes many benefits over prior art decontamination and/or sterilization methods. For example, the device allows rarely sterilized items, such as credit cards or paper money, to be easily decontaminated.

The disclosed device can easily slow or prevent the spread of harmful microorganisms, such as (but not limited to) bacterial infection, viruses (e.g., Influenza, COVID, etc.), fungi, mold, and the like.

Device 5 can be easily used by a wide variety of users, including children and the elderly.

The disclosed device can be safely used without harm to the user from excess exposure to ultraviolet radiation.

Device 5 is simple, compact, and easy to use.

The device is available as a stand-alone device or can be integrated into a larger system, such as an ATM or debit/credit card reader.

Advantageously, the device can be used in a single direction (input to exit or exit to input) or both directions, completing the disinfecting/sterilization process in a single pass through the device.

The device is configurable without or with a feed roller assist system, depending on the particular object to be disinfected or sterilized.

The device uses no chemicals, so is cleaner and safer compared to other prior art devices.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A device for sterilizing or disinfecting an object, the device comprising:
a housing defined by:
a front face and an opposing rear face;
a top face and an opposing bottom face;
a first side face and a second side face, wherein each side face includes a slot that provides access to the interior compartment; and
a pathway that extends from the first side face slot to the second side face slot within the interior compartment;
a plurality of ultraviolet bulbs positioned within the interior compartment of the housing, adjacent to the pathway.

2. The device of claim 1, wherein the device comprises a plurality of ultraviolet bulbs positioned above and below the pathway.

3. The device of claim 1, wherein the external surface of each slot includes a shielding mechanism.

4. The device of claim 1, wherein the interior compartment includes a coating that at least partially blocks or reduces ultraviolet radiation at or adjacent to the slots.

5. The device of claim 1, wherein the ultraviolet bulb emits radiation with a wavelength of about 100-400 nanometers.

6. The device of claim 1, wherein the ultraviolet bulb emits UV-A light, UV-B light, UV-C light, or combinations thereof.

7. The device of claim 1, wherein the ultraviolet bulb has a wavelength sufficient to kill about 80-100 percent of one or more viruses, bacteria, molds, or fungi.

8. The device of claim 1, wherein the interior compartment includes a reflective material to increase exposure of the ultraviolet bulb.

9. The device of claim 1, wherein at least one ultraviolet bulb is positioned above the pathway, below the pathway, or both.

10. The device of claim 2, wherein the plurality of bulbs are positioned in an arcuate pattern to at least partially surround the pathway on all sides.

11. The device of claim 2, wherein the plurality of bulbs include varying levels of ultraviolet light.

12. The device of claim 1, further comprising a plurality of rollers positioned directly above, below, or both above and below the pathway.

13. The device of claim 12, wherein the rollers are electromechanical rollers.

14. A method of decontaminating or sterilizing an object, the method comprising:
positioning the object within a slot of a decontaminating or sterilizing device, the device comprising:
a housing defined by:
a front face and an opposing rear face;
a top face and an opposing bottom face;
a first side face and a second side face, wherein each side face includes a slot that provides access to the interior compartment; and
a pathway that extends from the first side face slot to the second side face slot within the interior compartment;
a plurality of ultraviolet bulbs positioned within the interior compartment of the housing;
passing the object along the pathway of the device interior compartment, wherein the object is exposed to the ultraviolet bulbs on all sides;
removing the object from the opposing slot, wherein the object is decontaminated or sterilized on all sides.

15. The method of claim 14, wherein the object is passed along the pathway using one or more rollers positioned along the pathway.

16. The method of claim 14, wherein an output profile of each ultraviolet bulb is varied as desired by a user.

17. The method of claim 14, wherein the object can pass from the slot on the first side face to the slot on the second side face and vice versa.

18. The method of claim 14, wherein each ultraviolet bulb emits radiation with a wavelength of about 100-400 nanometers.

19. The method of claim 14, wherein each ultraviolet bulb emits UV-A light, UV-B light, UV-C light, or combinations thereof.

20. The method of claim 14, wherein each ultraviolet bulb has a wavelength sufficient to kill about 80-100 percent of one or more viruses, bacteria, molds, or fungi.
